(19) United States
(12) Patent Application Publication
(10) Pub. No.: US 2017/0258623 A1
(43) Pub. Date: Sep. 14, 2017

(54) Pre-Fabricated, Yet Highly Customizable Immobilization Device

(71) Applicant: Jennifer Lord, Austin, TX (US)

(72) Inventor: Jennifer Lord, Austin, TX (US)

(21) Appl. No.: 15/453,580

(22) Filed: Mar. 8, 2017

Related U.S. Application Data
(60) Provisional application No. 62/305,203, filed on Mar. 8, 2016.

(51) Int. Cl. A61F 5/058; A61F 13/04

(52) U.S. Cl. CPC A61F 5/058; A61F 13/04

(57) ABSTRACT

An immobilization device is provided herein that is pre-fabricated, yet highly customizable to the patient's body habitus and injury specifications without requiring an expert or a lengthy amount of time to create and custom fit the device. The pre-fabricated immobilization device described herein may comprise a polymer material that is moldable, re-moldable and reusable. Examples of such polymer materials include, but are not limited to, a thermoplastic material, a vitrimer material, or vitrimer-like material, each having a transition temperature ranging between about 100° F. and about 180° F., and in some embodiments, between about 110° F. and about 140° F. A method for fitting an immobilization device onto a patient is also provided herein.

This application claims priority to Provisional Patent Application No. 62/305,203, filed Mar. 8, 2016.

### BACKGROUND OF THE INVENTION

1. Field of the Invention

This invention relates to orthopedic immobilization devices and, more particularly, to orthopedic casts, splints and braces that are pre-fabricated, yet highly customizable.

2. Description of the Relevant Art

The following descriptions and examples are provided as background only and are intended to reveal information that is believed to be of possible relevance to the present invention. No admission is necessarily intended, or should be construed, that any of the following information constitutes prior art impacting the patentable character of the subjected mater claimed herein.

Fractures, soft tissue injuries, and burns are common occurrence in the extremities and the spine, and the majority of these injuries require immobilization. For appropriate extremity injury immobilization, a sturdy material is needed that will conform to the patient's body, but will not bend or allow motion of the injured body part. Depending on the type of injury, the immobilization device may be initially moldable, so that the physician can set the fracture or injury back into alignment, and then become rigid to hold the fracture or injured limb in the correct alignment. For fractures, it might take the physician several attempts to set the fracture or injured limb into the correct alignment.

The most commonly available immobilization devices for holding a new fracture or injury in correct alignment are plaster of Paris and fiberglass splints and casts. These materials are rolled on while wet and require an experienced technician or doctor to put on. Over the course of several minutes, the wet material dries and hardens. During that time, the limb or injury is placed into a corrected alignment. X-rays are generally taken through the cast and if the alignment of the fracture is judged to be not good enough, the cast is removed and discarded, and the process is repeated until the alignment is appropriately corrected. In some cases, this process may need to be repeated two or three times during a patient's visit. Each time the cast is removed and redone, it causes pain to the patient. There is also a potential for losing some of the correction in alignment each time the cast is removed and redone. This process may need to be repeated at multiple visits.

There are additional problems associated with plaster of Paris and fiber glass splints and casts. For example, the current materials used to form these devices are hot, do not breathe, may cause claustrophobia, and make it difficult to see the skin or any wounds under the splint or cast. If the liner used under the device is not waterproof, the patient will not be allowed to get the device wet for approximately 6-12 weeks, and this could lead to skin and odor issues. In some cases, contact pressure points may be created if a sprint or cast is applied inappropriately, or if significant swelling occurs after the splint or cast is applied. Contact pressure points can lead to nerve, muscle and soft tissue injury. Therefore, casts and splints that create such pressure points must be removed, discarded and replaced with new ones. Casts and splints made from plaster of Paris and fiber glass are also prone to cracking and breaking, and may be an issue for patients with claustrophobia, since they are formed circumferentially around the limb.

Pre-fabricated splints represent another form of commonly available immobilization device, and are typically made out of a neoprene material with metal stays, or a fabric and/or foam covered thermoplastic material. While each of these devices can be partially molded to adjust the alignment of the injury, they are generally not rigid enough to hold a fresh fracture in the corrected alignment and generally do not conform well enough to the injured body part to be the sole treatment for many injuries. Because most pre-fabricated splints come in discrete sizes with little to no chance for alteration, they are difficult to custom tailor to the individual patient and the injured body part, and lack support due to the fixed length of the prefabricated product. Many pre-fabricated splints include an under layer of cushioning and/or insulating material (e.g., neoprene, fabric or foam), which is bonded or permanently secured to a thermoplastic material, if used, on the side of the device adjacent to the patient's skin. As such, these devices tend to be hot, may or may not be waterproof, and are usually removed before getting the limb wet. They also tend to trap bacteria and mold in the under layer and have odor problems.

Custom-made splints can be created by occupational therapists by cutting a desired shape and size of splint out of flat sheets of thermoplastic material. After the desired shape is cut from the thermoplastic sheet, the cut shape is placed into a hot water bath to melt the thermoplastic and make it pliable enough for molding onto the patient. Although custom splints can be cut and molded to the patient's body and can hold an older injury relatively still, these devices require a lengthy period of time to create, require expert therapists to make them, and tend to be hot, bulky and heavy for patients to wear. In addition, these devices are not rigid enough, and do not conform well enough, to hold most new injuries in corrected alignment, and thus, are generally only used for older injuries that have already begun to heal.

3-D casts represent yet another form of immobilization device, which may be 3-D printed so as to conform to a scanned image of the patient's limb. While such devices may conform to the patient's exact specifications, these devices can take hours to print one device, and can take days to ship to the doctor for placement on the patient. Because of these limitations, 3-D casts are not appropriate for unstable fractures which require manipulation in the office. If these devices do not fit correctly or rub the skin, or if the fracture/injury is not in the corrected alignment, these devices must be thrown away and redone. 3-D casts do tend to be lightweight, allow the limb to get wet, and are good for those with claustrophobia, however, the cost and time needed to form such a device prohibits most doctors from using these devices.

A need exists for an immobilization device that overcomes the disadvantages of the currently known devices.

### SUMMARY OF THE INVENTION

The following description of various embodiments of an immobilization device is not to be construed in any way as limiting the subject matter of the appended claims.

Generally speaking, an immobilization device is provided herein that is pre-fabricated, yet highly customizable to the patient's body habitus and/or injury specifications without requiring an expert or a lengthy amount of time to create and custom fit the device. The pre-fabricated immobilization device described herein may be formed from a material that is moldable, re-moldable and reusable. In some embodiments, the material used to form the immobilization device may be inherently anti-fungal and/or anti-microbial. In other embodiments, an anti-fungal and/or anti-microbial coating may be applied to all or part of the immobilization device to reduce the likelihood of foul odor or disease.

According to one embodiment, the immobilization device may comprise a polymer material that can be converted from a rigid state to a pliable state by application of heat to the polymer material, so that the pliable polymer material can be molded directly onto the body part. To convert the polymer material from the rigid state to the pliable state, the heat applied to the polymer material may cause the temperature of the polymer material to exceed a transition temperature of the polymer material. In some embodiments, the polymer material may comprise a transition temperature, which may generally range between about 100° F. and about 180° F. More preferably, the transition temperature of the polymer material may range between about 100° F. and about 140° F.

After the transition temperature is exceeded and the polymer material becomes pliable, the pliable polymer material may be applied and molded onto the body part of the patient. In some cases, the polymer material may be allowed to cool slightly before the pliable polymer material is applied to avoid injury to the patient. In addition or alternatively, an under layer or liner, such as a compression garment, padding, or other insulating layer, may be arranged or fitted onto the body part to ensure patient comfort during the applying and molding processes. When cooled below the transition temperature, the polymer material may be converted back to the rigid state to immobilize the body part.

As used herein, a “transition temperature” is a temperature at which the polymer material transitions from a rigid state to a pliable state. Depending on the particular polymer material used to form the immobilization device, the transition temperature may be a glass transition temperature or a melt point temperature associated with the polymer material. As used herein, a “rigid state” means that the polymer material may comprise a tensile strength greater than or equal to about 45 megapascals (MPa) and an elastic modulus greater than or equal to about 1.5 gigapascals (GPa) when at a temperature below the transition temperature of the polymer material. Likewise, a “pliable state” means that the polymer material may comprise a tensile strength less than about 40 megapascals (MPa) and an elastic modulus less than about 1.0 gigapascals (GPa) when at a temperature at or above the transition temperature of the polymer material.

In some embodiments, the polymer material used to form the immobilization device may comprise a thermoplastic material, a vitrimer material or a vitrimer-like material. In one embodiment, the polymer material may be an acrylonitrile butadiene styrene (ABS) material, which has been modified to exhibit a glass transition temperature ranging between about 100° F. and about 140° F. In one example, the modified ABS material may be synthesized with an alkyl-substituted styrene to achieve a glass transition temperature within the desired range. However, the immobilization device is not limited to a modified ABS material, and may alternatively comprise other polymer materials with glass transition temperatures within the desired range.

Heat may be applied to the polymer material in a variety of different ways including, but not limited to, application of wet heat (e.g., hot water baths or steam), application dry heat (e.g., hot air), irradiation (e.g., microwave energy, ultrasound energy or light energy), or heat produced by electricity (e.g., by supplying current to electrical wires embedded within the polymer material). In some embodiments, provisions can be made for protecting the patient's skin from excess heat transfer. In one example, an underside of the immobilization device adjacent to the patient's skin may be coated with an insulating material (e.g., silicon or rubber) to reduce the amount of heat transmitted to the patient's skin. In another example, an under layer or liner, such as a compression garment, padding, or other insulating layer, may be arranged or fitted onto the body part before the material is molded onto the injured body part to protect the patient's skin from excess heat. In some embodiments, the under layer or liner may be removed from the patient after the material has cooled and set.

After conforming the immobilization device onto the body part of the patient, the polymer material may be allowed to cool and harden back to the rigid state to immobilize the body part and/or hold new injuries and fractures in their corrected alignment. However, it is generally desirable that the polymer material be re-moldable to allow the alignment to be re-corrected, to account for swelling of the injured body part, and/or to reduce contact pressure points. In some embodiments, heat may be reapplied to the polymer material while the immobilization device remains on the patient to convert the polymer material back to the pliable state and allow the device to be re-molded onto the body part. This may cause less pain for the patient if the immobilization device needs to be adjusted, as compared to a device that has to be removed and redone. In other embodiments, however, the immobilization device may be remolded by removing the immobilization device, applying heat to the polymer material to convert the polymer material back to the pliable state, and re-molding the polymer material directly onto the body part. Since the polymer material is re-moldable and resistant to bacteria and other disease causing agents, the immobilization device may be reused for a new injury.

The immobilization device described herein may also be lightweight, waterproof, and may comprise one or more large openings that reduce the weight of the device and allow airflow and access to the skin. In some embodiments, the one or more large openings may range between about 2 mm to about 150 mm in diameter. In addition, the immobilization device may comprise one or more stabilization regions of solid polymer material, which may be arranged adjacent to the body part to be immobilized (e.g., for holding an injury or fracture in the corrected alignment and/or for providing increased protection to the body part or injured area). In some embodiments, the one or more stabilization regions may each include an area of solid polymer material ranging between about 1 inch to about 4 inches in width, and ranging between about 2 inches to about 16 inches in length, depending on the body part to be immobilized. In some embodiments, the immobilization device may be non-circumferential, meaning that it may not completely wrap around the patient's limb or torso. This may be helpful for patients that suffer from claustrophobia and may also allow for swelling.

Different embodiments of the immobilization device described herein may be provided in different sizes and may be designed to accommodate different body parts, such as a wrist and/or hand, an arm, an ankle and/or foot, a leg, a torso, a head, etc. In some embodiments, the immobilization device may be designed to hold and/or reshape a patient's cranium. In some embodiments, the immobilization device may be reversible, in that the same device may be configured for placement on either the right or left limb.

The immobilization device described herein may also be highly customizable to the patient and/or the patient's injury. In some embodiments, a plurality of small openings and/or perforations may extend along the entire periphery of the device, including the length, width and ends, so that the device can be trimmed to decrease the length or the girth of the device, or to decrease rubbing on certain areas of the patient's skin. In some embodiments, each of the small openings may range between about 3 mm to about 30 mm in length and about 2 mm to about 15 mm in width. In some embodiments, the small openings may be spaced from each other by about 2 mm to about 15 mm. In some embodiments, the immobilization device may comprise several rows of these small openings and/or perforations, so that one or more rows remain intact, even if the device is trimmed to custom fit the device to the patient.

In some embodiments, the immobilization device may be customizable by trimming one or more edges, or removing portions of the device, to tailor the device to a right limb or a left limb, or to convert the immobilization device from one type to another. In one example, lower edges of the immobilization device can be trimmed to convert a muenster splint into a short arm splint. In another example, a long leg splint can be converted into a short leg splint by trimming upper edges of the immobilization device. In another example, an immobilization device initially configured for immobilizing the wrist, hand and fingers can be shortened into a hand and finger splint. In another example, an immobilization device initially configured as an ulnar gutter hand splint may be converted to a clam digger splint by removing an upper right portion or an upper left portion of the device.

In some embodiments, the immobilization device may also be customizable by using some of the small openings extending along the periphery of the device for attaching fasteners at multiple places along the periphery of the device to fasten the immobilization device onto the patient. Since the small openings extend along the entire periphery of the device, a multitude of attachment locations may be available for securing the device and creating different force vectors on the injured body part.

According to another embodiment, a method for fitting an immobilization device onto a patient may generally begin by obtaining an immobilization device having a pre-fabricated shape, which is rigid, yet configurable to conform to a body part of the patient through application of heat to the immobilization device. In some embodiments, the immobilization device may be obtained in a flat form to reduce the inventory space needed to store such devices. In other embodiments, the immobilization device may be obtained in a rolled form to reduce inventory space.

As noted above, the immobilization device may comprise a polymer material having a transition temperature ranging between approximately 100° F. and approximately 180° F., in some embodiments. In other embodiments, the transition temperature of the polymer material may range between approximately 100° F. and approximately 140° F. In order to convert the polymer material to a pliable state, the method may include applying heat to the immobilization device to increase a temperature of the polymer material past the transition temperature. In some embodiments, the step of applying heat to the immobilization device may include placing the immobilization device in a hot water bath, a convection oven or a microwave oven for a length of time and temperature sufficient to convert the polymer material to the pliable state. In other embodiments, the step of applying heat to the immobilization device may comprise using a hot air gun, an ultrasound source, an electrical source or a light source to apply heat to the immobilization device for a length of time and temperature sufficient to convert the polymer material to the pliable state.

While the polymer material is in the pliable state, the method may further include applying the pliable polymer material onto the patient, conforming the immobilization device to the body part and subsequently allowing the polymer material to cool and harden back to a rigid state to immobilize the body part. In some embodiments, the method may allow the pliable polymer material to cool slightly before applying the pliable polymer material onto the patient, so as to avoid injuring the patient. In other embodiments, the method may include arranging or fitting an under layer or liner (such as a compression garment, padding, or other insulating layer) onto the body part, before the pliable polymer material is applied, to ensure patient comfort during the applying and conforming steps.

In some embodiments, the method may include determining whether adjustment is needed in at least a portion of the immobilization device (e.g., to correct an alignment of an injury) after the polymer material has cooled and hardened back to the rigid state to immobilize the body part. If adjustment is determined to be needed, the method may continue by reapplying heat to at least the portion of the immobilization device needing adjustment to convert the polymer material back to the pliable state, reforming the immobilization device and subsequently allowing the polymer material to cool and harden back to the rigid state. In some embodiments, heat may be reapplied to the immobilization device while the device remains on the patient. For example, heat may be reapplied using a hot air gun, an ultrasound source, an electrical source or a light source to reapply heat locally to at least the portion of the immobilization device determined to need adjustment. In other embodiments, the immobilization device may be removed from the patient before heat is reapplied to reform the immobilization device.

As noted above, the immobilization device may comprise one or more rows of small openings extending along an entire periphery of the immobilization device. In some embodiments, the method may comprise tailoring the immobilization device to fit a particular injury or patient by cutting away one or more of the rows of small openings. In some embodiments, the method may further comprise using a plurality of the small openings for attaching fasteners at multiple places along the periphery of the immobilization device to fasten the immobilization device onto the patient.

### BRIEF DESCRIPTION OF THE DRAWINGS

Other objects and advantages of the invention will become apparent upon reading the following detailed description and upon reference to the accompanying drawings.

FIG. 1 is a top view of a first embodiment of an immobilization device prior to application of the device onto a patient;

FIG. 2 is a view of the immobilization device shown in FIG. 1 after application of the device onto the patient;

FIG. 3 is a magnified view of a portion of the immobilization device shown in FIG. 1 illustrating in greater detail certain features of the immobilization device;

FIG. 4 is a top view of a second embodiment of an immobilization device prior to application of the device onto a patient;

FIG. 5 is a top-side view of the immobilization device shown in FIG. 4 after application of the device onto the patient;

FIG. 6 is a bottom-side view of the immobilization device shown in FIG. 4 after application of the device onto the patient;

FIG. 7 is a top view of a third embodiment of an immobilization device prior to application of the device onto a patient;

FIG. 8 is a view of the immobilization device shown in FIG. 7 after application of the device onto the patient, further illustrating how the same device may be placed on either the right or left limb;

FIGS. 9A and 9B are front and back views of a fourth embodiment of an immobilization device after application of the device onto a patient;

FIGS. 10A and 10B are front and back views of a fifth embodiment of an immobilization device after application of the device onto a patient;

FIG. 11 is a front view of a sixth embodiment of an immobilization device after application of the device onto a patient; and

FIG. 12 is a front view of a seventh embodiment of an immobilization device after application of the device onto a patient.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

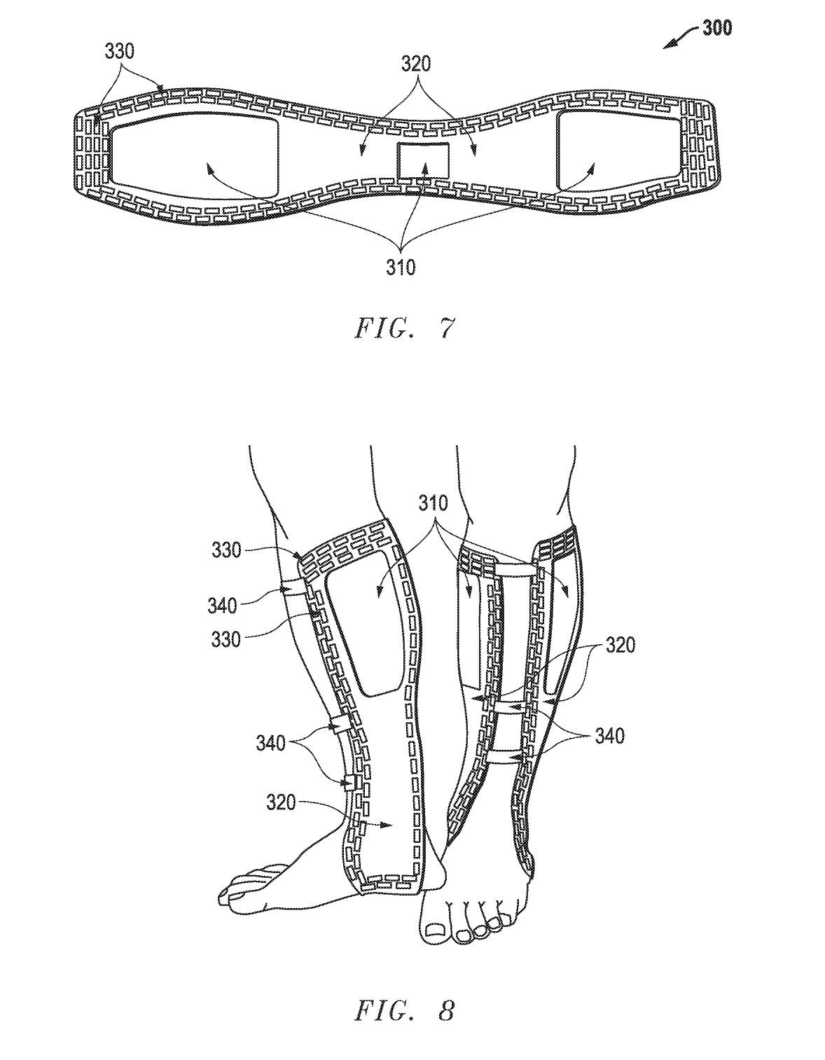

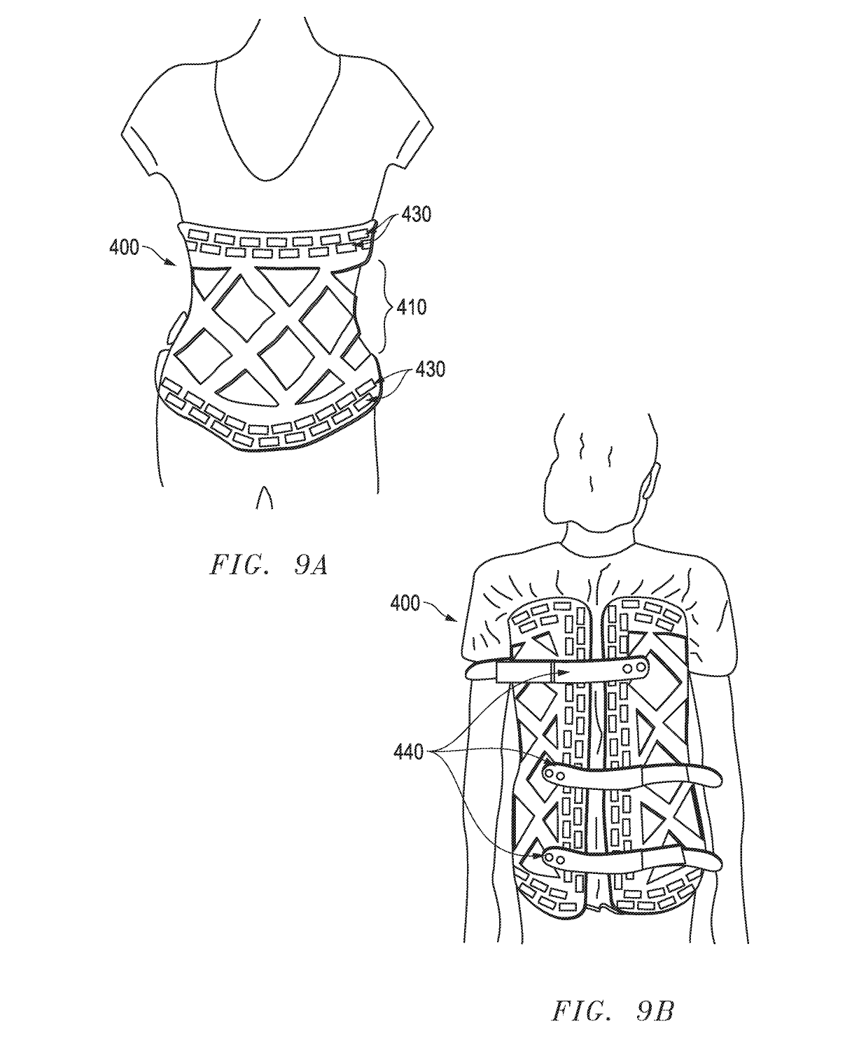

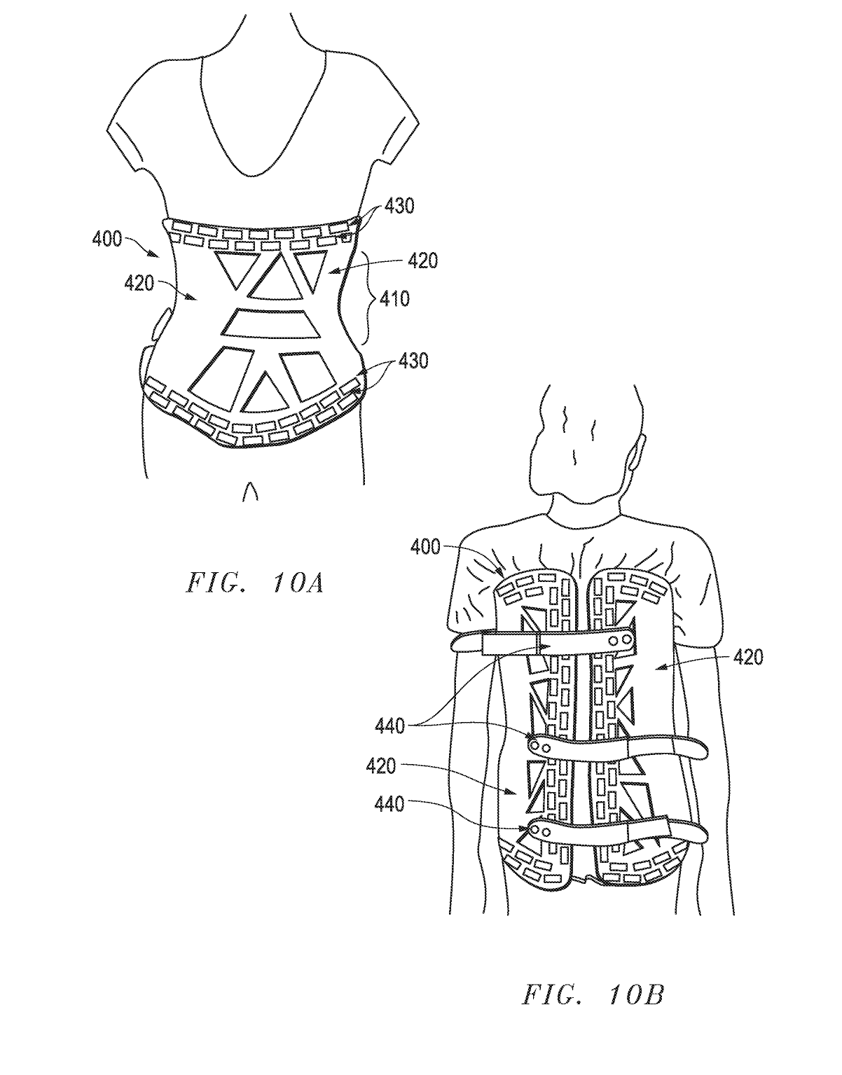

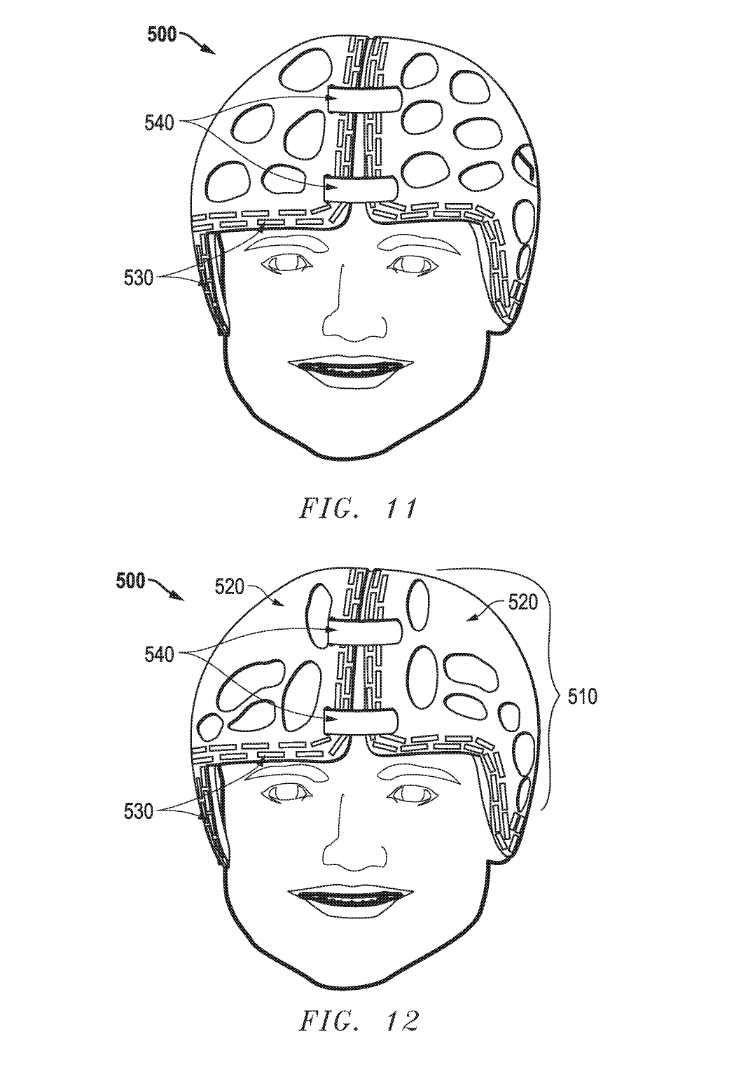

Turning now to the drawings, FIGS. 1-12 illustrate various embodiments of an immobilization device that is pre-fabricated, yet highly customizable to the patient's body habitus and injury specifications without requiring an expert or a lengthy amount of time to create and custom fit the device. FIGS. 1-3 illustrate a first embodiment of the immobilization device 100 as an arm and/or forearm splint. FIGS. 4-6 illustrate a second embodiment of the immobilization device 200 as a hand and/or wrist splint. FIGS. 7-8 illustrate a third embodiment of the immobilization device 300 as an ankle and/or leg splint. FIGS. 9A-9B and 10A-10B illustrate a fourth and fifth embodiment of the immobilization device 400 as a torso splint or back brace used, for example, to treat scoliosis. FIGS. 11-12 illustrate a sixth and seventh embodiment of the immobilization device 500 as a helmet used, for example, to reshape a child's cranium. Although the embodiments shown in FIGS. 1-3, 4-6, 7-8, 9-10 and 11-12 may generally differ from one another in size, shape and location of intended use, they contain many of the same or similar features. Similar features are represented with like numerals in the drawings.

As shown most clearly in FIGS. 1, 3, 4 and 7, the immobilization device described herein is pre-fabricated in a shape particular to a specific part of the body that it is intended to immobilize, yet provided to the customer in a substantially flat form. In some cases, various sizes of the pre-fabricated immobilization device may be provided for each shape/body part, so as to accommodate different sizes and/or ages of patients. By providing the pre-fabricated immobilization device to the customer in the flat form shown, for example, in FIGS. 1, 3, 4 and 7, the inventory space needed to store such devices is reduced compared to other pre-fabricated immobilization devices that are provided to the customer as a three-dimensional structure.

In general, the various embodiments of immobilization devices described herein may be formed from substantially any polymer material that is pliable at temperatures ranging between approximately 100° F. and approximately 180° F., yet substantially rigid at temperatures below approximately 100° F. In some embodiments, the immobilization device described herein may be formed via an extrusion, injection molding, compression molding or 3-D printing process, or may be stamped or cut from a flat sheet of polymer material to form the desired shape and size. In some embodiments, the immobilization devices described herein may be formed in a two-step fabrication process, whereby a molding process is used to form the overall shape and size, and a cutting or stamping process is used to create certain features of the immobilization device. Regardless of the particular fabrication process used, the end-result of the fabrication process may be a flat rigid form, as shown in the exemplary immobilization devices depicted in FIGS. 1, 3, 4 and 7. Alternatively, the immobilization device may be provided to a user in a rolled form.

According to one embodiment, the polymer material used to form the immobilization device may be a thermoplastic copolymer or polymer blend. In some embodiments, the thermoplastic copolymer or polymer blend may have a transition temperature point (e.g., the point at which a solid material begins to soften) ranging between approximately 100° F. and approximately 180° F. In some embodiments, the immobilization device is preferably formed from a thermoplastic polymer having a transition temperature ranging between approximately 100° F. and approximately 140° F. The transition temperature may be a glass transition temperature (T_{g}) or a melt point temperature (Tₘ), depending on the particular thermoplastic polymer used to form the immobilization device. Examples of thermoplastic materials having suitable transition temperatures include, but are not limited to, acrylonitrile butadiene styrene (ABS) with or without an additive, polycaprolactone (PCL) with or without an additive, and polylactic acid (PLA) with or without an additive. Other thermoplastic materials with suitable glass transition temperatures may also be used.

In one particular embodiment, the immobilization device may comprise a modified acrylonitrile butadiene styrene (ABS) material. As known in the art, ABS is typically synthesized from a mixture of styrene and acrylonitrile in the presence of polybutadiene or a butadiene copolymer, and is a light-weight, non-crystalline (amorphous) material that exhibits high impact resistance and mechanical toughness. ABS is also known to exhibit a high molar mass of greater than 200 g/mol, an elastic modulus of about 2 gigapascals (GPa), and in some grades, a tensile strength of about 50 megapascals (MPa) or more. The combination of high elastic modulus and tensile strength ensures that ABS is very tough at temperatures below its glass transition temperature.

Although ABS is commercially available in a number of different grades, each having its own chemical, mechanical and electrical properties, the glass transition temperature of commercially available ABS materials typically exceeds 220° F., rendering such materials unsuitable for the immobilization device described herein. However, the present inventor has recognized that by replacing the styrene typically used in the ABS synthesis procedure with an alkyl-substituted styrene, a new ABS material can be produced with a glass transition temperature (Tg) ranging between approximately 110-140° F. According to one embodiment, the new ABS material may comprise a tensile strength greater than or equal to about 45 megapascals (MPa) and an elastic modulus greater than or equal to about 1.5 gigapascals (GPa) when at a temperature below the glass transition temperature of the material. Likewise, the new ABS material may comprise a tensile strength less than about 40 megapascals (MPa) and an elastic modulus less than about 1.0 gigapascals (GPa) when at a temperature at or above the glass transition temperature of the material. In at least one embodiment, a modified ABS material is preferred, since it would enable the immobilization device to achieve a pliable state at relatively low temperatures, which may not injure or cause the patient significant discomfort. It is noted, however, that the immobilization device described herein is not limited to a modified ABS material, and may alternatively comprise other thermoplastic materials with glass transition temperatures within the desired range.

According to another embodiment, the polymer material used to form the immobilization device may comprise a vitrimer or vitrimer-like material, which has a glass transition temperature (T_{g}) point ranging between approximately 120° F. and approximately 180° F. According to one embodiment, the vitrimer material may comprise a tensile strength greater than or equal to about 45 megapascals (MPa) and an elastic modulus greater than or equal to about 1.5 gigapascals (GPa) when at a temperature below the glass transition temperature of the material. Likewise, the vitrimer may comprise a tensile strength less than about 40 megapascals (MPa) and an elastic modulus less than about 1.0 gigapascals (GPa) when at a temperature at or above the glass transition temperature of the material. Examples of suitable vitrimer materials include, but are not limited to, a polyimine vitrimer with or without an additive, a polylactide vitrimer with or without an additive, and an epoxy-based vitrimer with or without an additive. Examples of polyimine vitrimers suitable for use in the immobilization device described herein are disclosed in U.S. Pat. No. 9,453,099, the entirety of which is incorporated herein by reference. However, one skilled in the art would understand how other vitrimer and vitrimer-like materials may be used to form the immobilization device described herein without departing from the scope of the invention.

In some embodiments, a thermoplastic polymer, vitrimer or vitrimer-like material may be reinforced with an additive to modify an electrical, mechanical or thermal property of the material. Examples of additives that may be combined with a thermoplastic, vitrimer or vitrimer-like material include, but are not limited to, carbon fibers or carbon nanotubes, a lingo-cellulose additive, Kevlar, glass and graphite. In one example, carbon fibers or carbon nanotubes may be added to a thermoplastic material to increase a tensile and flexural strength and decrease an electrical resitivity of the material. However, as this may also increase the transition temperature of the combined material, an additive may not be desirable in all embodiments.

Thermoplastic and vitrimer materials are polymer materials that are generally pliable at temperatures above, and rigid at temperatures below, their glass-transition temperatures (T_{g}). Once heated above their T_{g} point, these materials can be molded into forms that conform to a patient's body habitus and/or injury specifications. Once cooled below their T_{g} point, these materials begin to harden and become more rigid. In some cases, the rigidity of the material may be sufficient to hold a fracture in the corrected alignment once the pliable polymer material has cooled below the T_{g} point. However, adjustment may sometimes be needed to allow a fracture alignment to be re-corrected, to account for swelling of an injured body part, to reduce contact pressure points, and/or to heal any cracks that may have developed in the immobilization device. If adjustment is needed, the polymer material can be re-heated, in whole or in part, past the glass-transition temperature (T_{g}) to re-mold or reform all or part of the immobilization device.

The polymer material can be heated and re-heated in a variety of different ways. For example, if molding the device onto the patient for the first time, or if the entirety of the device needs to be reformed, the device may be placed in a hot water bath, a convection oven or a microwave for a length of time and temperature sufficient to make the material sufficiently pliable. The length of time and temperature generally depends on the method used to heat the polymer material, and the transition temperature of the polymer material, and thus, will differ for different methods and materials. In some cases, the immobilization device may be adjusted after the device is molded onto the patient by applying only localized heat to certain areas of the device. For example, a hot air gun, irradiation source (e.g., an ultrasound source or a light source) and/or an embedded electrical source may be used to heat certain areas of the device past the T_{g} point, so that these areas may be re-molded. In some embodiments, certain areas of the device may be re-molded while the device remains on the patient. This may be beneficial to the patient, as it may cause less pain than if the device had to be removed and redone. The method of heating used to soften the polymer material determines whether or not the material can be reheated and remolded while remaining on the patient. Examples of heating methods that may be used while the device remains on the patient include application of hot air, irradiation (such as an ultrasound or light source), and/or heat generated by an embedded electrical source.

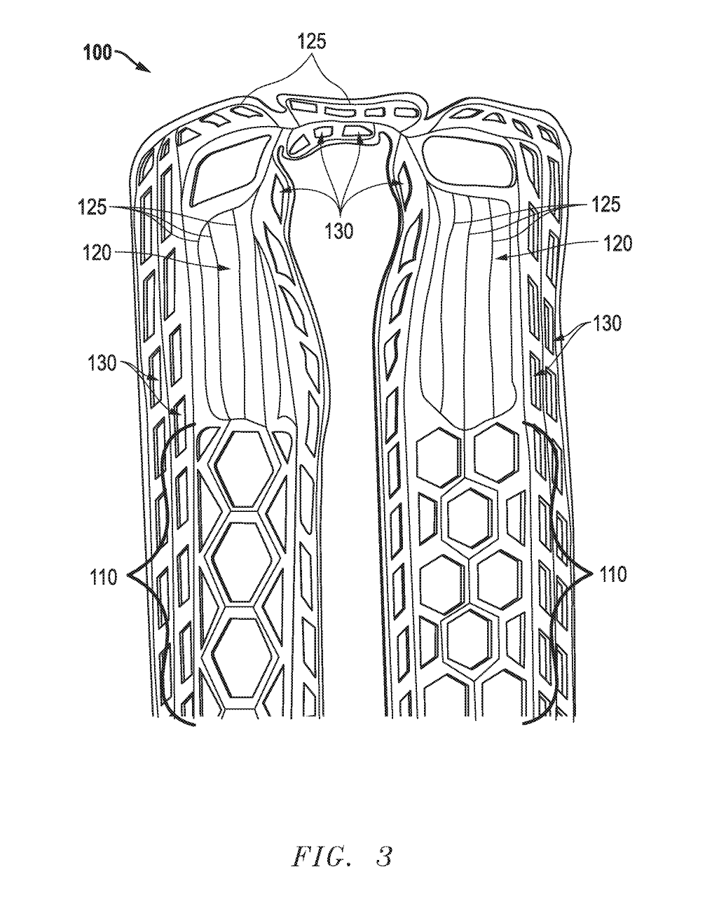

In some embodiments, the immobilization devices described herein may have bare electrical wires embedded within the polymer material used to form the device. These wires may be embedded within all or part of the device, and may extend out from the periphery or top surface of the device for connection to an electrical source, such as a battery or A/C outlet. FIG. 3 illustrates an exemplary arrangement of electrical wires 125, which are embedded within the polymer material used to form immobilization device 100. It is understood that a similar or different arrangement of electrical wires could also be embedded within the immobilization devices shown in FIGS. 1-2 and 4-12. Alternatively, one or more of the immobilization devices shown in FIGS. 1-12 may use an alternative heat source for heating and/or re-heating the polymer material.

In some embodiments, electrical current may be applied to the electrical wires 125 for heating all or part of the device past the glass-transition (T_{g}) point. In some embodiments, sections of the device may have separate electrical circuits for selectively heating one or more sections of the device, while not heating other sections, so that certain areas of the device can be reheated and adjusted. For example, adjustment may be needed to relieve pressure on the web space of the hand, or over bony prominences, while still allowing the material to hold the injury in a reduced position, so that the alignment and reduction is not lost. In another example, adjustment may be needed to re-correct the alignment in the fractured area, while leaving the rest of the device intact and hard. By embedding separate electrical circuits in one or more sections of the device, certain areas of the device can be quickly and easily adjusted by applying electrical current to the wires embedded in only those section(s). In some embodiments, a conductive additive (e.g., carbon fibers) may be added to the polymer material used to form the immobilization device to improve electrical conductivity throughout the material.

In some embodiments, a two-dimensional sheet of conductive material (e.g., silver, copper, aluminum, tungsten, etc.) may be embedded within, or sandwiched between, two layers of the polymer material to provide an electrically conductive layer, as opposed to a circuit or network of wires. While this may provide more even heating over one or more areas of the device, it may limit the fabrication method used to form the device. For example, such a device may be formed by bonding the two-dimensional sheet of conductive material between two sheets of the polymer material and then cutting or stamping the desired shape and size out of the bonded layers.

In some embodiments, one or more exterior surfaces of the immobilization device may be coated with one or more materials to prevent sticking or tackiness when the polymer material is in the pliable state, to reduce the likelihood of foul odor or disease, and/to reduce the amount of heat transfer to the patient. For example, one or more exterior surfaces of the immobilization device may be coated with silicon or another non-stick material (e.g., a flouropolymer coating, such as Xylan®) to prevent sticking or tackiness in the moldable state. To reduce the likelihood of foul odor or disease, an anti-fungal and/or anti-microbial coating may also be applied to one or more exterior surfaces of the immobilization device, in some embodiments. To reduce heat transfer to the patient, silicon, rubber, urethane/polymer foam or another insulating material may be used, in some embodiments, to coat one or more exterior surfaces of the immobilization device. The coating material(s) may be applied using any known method(s).

In some embodiments, an under layer or liner, such as a compression garment, foam padding, or other insulating layer, may be arranged or fitted onto the injured body part before the polymer material is heated and molded onto the injured body part to further protect the patient's skin from excess heat. This under layer or liner may be used as a primary heat protection means if the exterior surface(s) are not coated with an insulating material, or as a secondary heat protection means if an insulating coating is included. In some cases, the under layer or liner may be removed from the patient after the polymer material has cooled and hardened back to the rigid state. In other cases, the under layer or liner may remain, or may be replaced by another under layer or liner, to enhance patient comfort, to aid in wound healing, or to provide additional protection and/or padding to a particular area being immobilized.

Returning to the drawings, the immobilization devices described herein may include many desirable features, which reduce the weight of the device, while providing rigidity and support at the location of injury, and a highly customizable fit. As shown in FIGS. 1-10, for example, the immobilization device 100, 200, 300, 400, and 500 may comprise one or more areas 110, 210, 310, 410, and 510 having one or more large openings that reduce the weight of the device and allow airflow and access to the skin. These large openings may be formed during the injection or compression molding process, or may be stamped or cut from the device sometime during or after the overall shape and size of the device is formed. In some embodiments, the large openings may range in size from about 2 mm to about 40 mm in diameter. In other embodiments, substantially larger openings ranging from about 15 mm to about 150 mm in diameter may be included. In addition to eliminating excess material, weight and bulk, the large openings included within the one or more areas 110, 210, 310, 410, and 510 may increase airflow to the patient's skin, allow for skin and wound monitoring and decrease the likelihood of claustrophobia. In some cases, the large openings may also enable a patient to more easily wash the skin under the immobilization device without having to first remove the device, and may allow the patient to more easily scratch an itch without using a dangerous utensil (like a coat hanger or pencil).

If more than one large opening is provided in the areas 110, 210, 310, 410, and 510, the plurality of large openings may be substantially random in nature, or may comprise a repeating pattern of geometrical shapes. As shown in FIG. 3, for example, immobilization device 100 includes an area 110 of large openings having a honeycomb pattern and shape. In FIGS. 9A-9B and 10A-10B, immobilization device 400 includes an area 410 of large openings having a repeating pattern of diamond shapes. In FIGS. 11-12, immobilization device 500 includes an area 510 of large, substantially circular or elliptical shaped openings, which may or may not extend over a majority of the immobilization device. Although only one large opening is illustrated in areas 110, 210 and 310 of the immobilization devices 100, 200 and 300 shown in FIGS. 1-2, 4-6 and 7-8, it should be understood that these areas may alternatively include a plurality of large openings having any of the shapes, patterns and sizes shown and described herein. It should also be understood that other shapes, patterns and sizes not specifically mentioned herein may also be used within any of the immobilization devices described herein.

In some embodiments, rigidity and support at the location of injury or correction may be provided by including stabilization regions 120, 220, 320, 420 and 520 of solid material in one or more of the immobilization devices described herein. The stabilization regions 120, 220, 320, 420 and 520 may be otherwise referred to as correction/stabilization bars, and may be generally configured for holding a fracture in the corrected alignment, and/or for providing increased protection to the fractured, injured or corrected area. As such, it is generally desired that the stabilization regions be dimensioned to achieve such purpose. For example, the stabilization regions 120, 220, 320, 420 and 520 may comprise a relatively large area of solid material (i.e., no openings or holes) ranging between about 1 inch and about 4 inches in width, and further ranging about 2 inches and about 16 inches in length, depending on the body part being immobilized. The exact size and shape of the stabilization region 120, 220, 320, 420 and 520 may generally depend on the form factor, or the intended use of the immobilization device. For example, the stabilization regions 320, 420 and 520 included within ankle/leg splint 300, torso splint 400 and helmet 520 may be significantly larger than the stabilization region 220 intended to immobilize the wrist, hand or fingers in hand splint 200.

An immobilization device that combines stabilization regions 120, 220, 320, 420 and 520 with one or more areas 110, 210, and 310 of large openings has been found to provide a particularly good compromise between patient comfort and rigidity and support at the location of injury. However, it may not be necessary to include stabilization regions in all embodiments of the immobilization devices described herein. For example, stabilization regions may or may not be included within the torso splint 400 or helmet 500, as shown in FIGS. 9-12.

If included, the stabilization regions may have any shape and size desired, and may be located in common areas for fracture or injury. As shown in FIGS. 1-3, for example, an immobilization device 100 configured as a muenster splint may have one or more stabilization regions 120 of substantially solid material that function to reinforce or protect the distal forearm and wrist, including the distal radius and ulna, carpal bones, proximal metacarpals, and distal radioulnar joint. As shown in FIGS. 4-6, an immobilization device 200 configured as an ulnar gutter hand splint or a clam digger splint may have one or more stabilization regions 220 of substantially solid material that function to reinforce or protect the hand and fingers, including the metacarpals and phalanges. Likewise, the immobilization device 300 shown in FIGS. 7-8 may have stabilization regions 320 adjacent to the tibia, fibula, ankle and subtalar joints, and tarsal bones.

In some embodiments, the stabilization regions may be formed as an integral component of the immobilization device by fabricating the device with relatively large area(s) of solid material. In some embodiments, a separate stabilization bar (or bars) may be added to the immobilization device in addition to, or in place of, the stabilization regions shown and described in the figures. If separate stabilization bar(s) are included, they may be fused to the immobilization device during the initial molding process used to fabricate the device, or the subsequent molding process used to reheat the polymer material, so that the device can be molded onto the patient.

The immobilization devices 100, 200, 300, 400 and 500 shown in FIGS. 1-12 have several features, which enable the pre-fabricated devices to be customized to the patient and the patient's injury. For example, the immobilization devices shown and described herein may include a plurality of small openings and/or perforations 130, 230, 330, 430 and 530, which extend along the periphery of the device. The small openings and/or perforations enable the device to be trimmed (e.g., to customize the fit to a particular patient and/or to reduce rubbing or contact pressure points) and also provide a plurality of attachment locations for securing the device with fasteners (such as Velcro or elastic straps). In some embodiments, the small openings may range between about 3 mm to about 30 mm in length, about 2 mm to about 15 mm in width, and may be separated from each other by about 2 mm to about 15 mm. In some embodiments, the small openings may have a substantially rectangular or parallelogram shape. In some embodiments, at least some of the small openings may have a shape with one or more curved sides, which mimics a contour of the peripheral edge adjacent to the small opening.

In some embodiments, the small openings and/or perforations 130, 230, 330, 430 and 530 may extend along the entire periphery of the immobilization device, including the length, width and ends, so that the device can be trimmed to decrease the length and/or the girth of the device, and/or to decrease rubbing on certain areas of the patient's skin. In some embodiments, the immobilization device may comprise several rows of these small openings and/or perforations, so that one or more rows remain intact, even if the device is trimmed to custom fit the device to the patient. In some embodiments, certain areas of the device may include more rows than other areas of the device. As shown in FIGS. 1 and 3, for example, a lower end of the immobilization device 100 may comprise a larger number (e.g., 3) of rows of openings 130 than the number of rows (e.g., 1-2) included at an upper end of the immobilization device 100. This may be because, in a muenster splint type device, it is generally more desirable to trim the lower end of the splint (i.e., the end closest to the elbow) to account for variability in size of forearm musculature. Likewise, it is generally more desirable to trim the upper end of the leg/ankle splint 300 to account for calf size variability.

In some embodiments, the immobilization device may be customized by trimming one or more edges of the device, to tailor the device to a particular injury or patient. For example, lower edges of immobilization device 100 (FIGS. 1-3) can be trimmed to convert a muenster splint into a short arm splint by cutting away one or more rows of the small openings 130 extending along the lower edges. Likewise, an immobilization device 200 (FIGS. 4-6) initially configured for immobilizing the wrist, hand and fingers can be shortened into a hand and finger splint by cutting away one or more rows of small openings 230 extending along the lower edges. In addition, a long leg splint can be converted into a short leg splint by trimming one or more rows of small openings 330 along the upper edges of the immobilization device 300 (FIGS. 7-8).

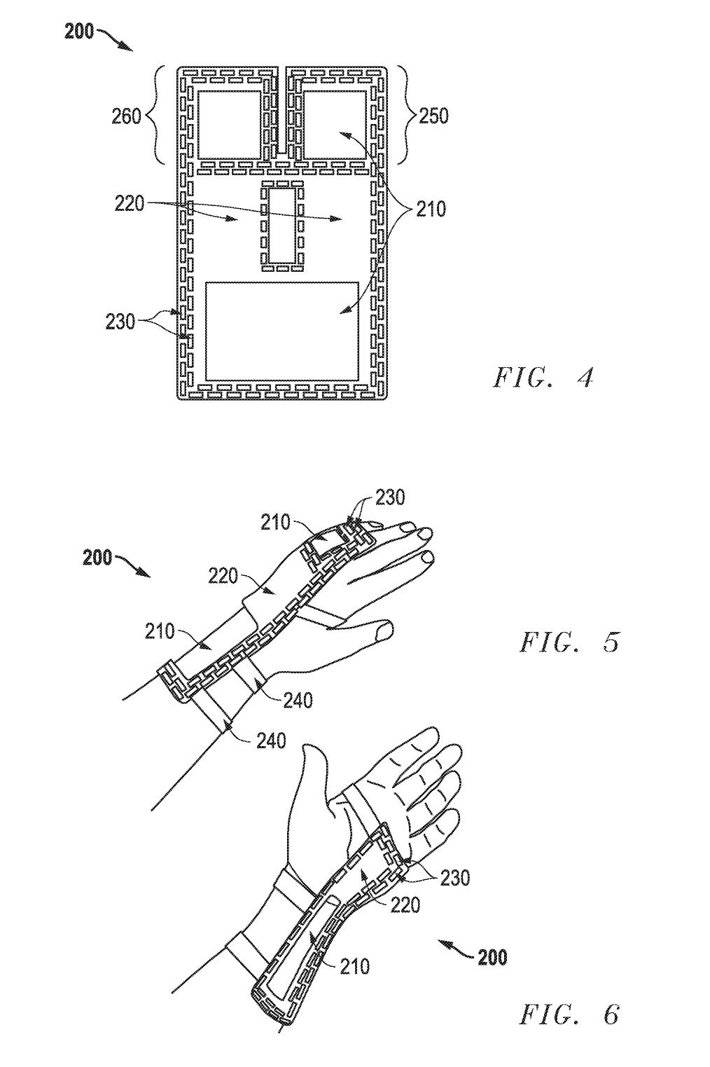

In some embodiments, the immobilization device may be further customized by removing one or more portions of the device to tailor the device to a right limb or a left limb, or to convert the device from one type to another. For example, an immobilization device 200 initially configured as an ulnar gutter hand splint (FIG. 4) may be converted to a clam digger splint for the left hand (FIGS. 5-6) by removing an upper right portion 250 of the device, or a clam digger splint for the right hand (not shown) by removing an upper left portion 260 of the device. Some immobilization devices, however, such as the ankle splint device 300 shown in FIGS. 7-8, may require little to no trimming to fit the device to the right or left limb.

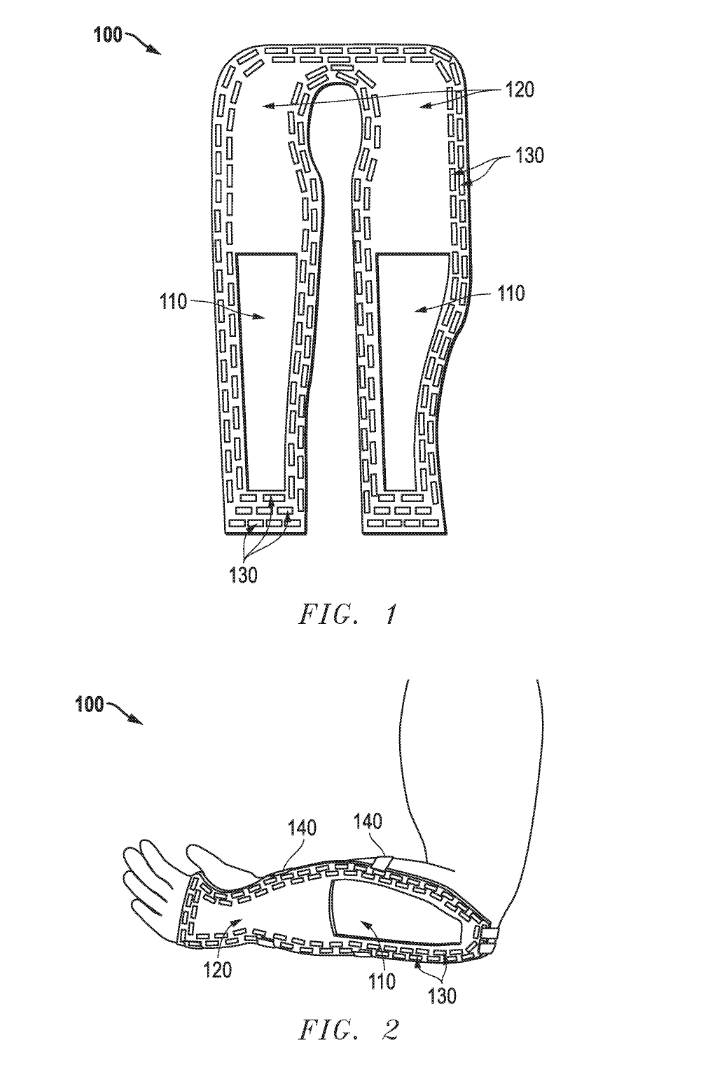

In some embodiments, the immobilization devices may be further customized by using some of the small openings 130, 230, 330, 430 and 530 extending along the periphery of the device for attaching fasteners 140, 240, 340, 440 and 540 at multiple places along the periphery of the device. Even if the immobilization device is trimmed, a multitude of attachment locations may still be available for securing the device and creating different force vectors on the injured body part. FIGS. 2, 5, 6, 8, 9B, 10B, 11 and 12 illustrate exemplary attachment locations for attaching a plurality of fasteners 140, 240, 340, 440 and 540 at multiple places along the periphery of the device. Although particular locations are shown in these figures, any of the small openings 130, 230, 330, 430 and 530 extending along the periphery of the device may be used for this purpose. In addition, various types of fasteners may be used including, but not limited to, hook and loop fasteners (e.g., Velcro™ straps), elastic fabric with clips, or elastic string. The fasteners may be placed anywhere along the periphery of the device to customize the stability of the device and may be loosened and tightened as needed.

Due to the polymer material used to form the immobilization devices described herein, the devices are radiolucent (allow x-rays to be taken through the device) and waterproof and may be worn while showering, bathing or swimming. However, the immobilization devices described herein may also be used with different splint liners depending on the needs of the patient and the area being immobilized. For example, the immobilization devices described herein may be used with a foam padding, a waterproof liner, a compression garment, over surgical dressings, over clothing, or even on bare skin.

A method for fitting an immobilization device onto a patient is also provided herein. In some cases, the method may begin by placing or fitting an appropriate splint liner on the injured body part or desired area for immobilization. However, a splint liner may not be necessary and may be omitted in other embodiments. Regardless of whether a splint liner is used, an immobilization device of the appropriate shape and size may be selected for the particular injury or area to be immobilized on the patient. As noted above, the immobilization device may be obtained in a flat form prior to application onto the patient, as shown in FIGS. 1, 3, 4 and 7, or in a rolled form (not shown). Next, the immobilization device may be heated to soften the polymer material (e.g., the thermoplastic or vitrimer material). As noted above, the immobilization device may be heated in a variety of different ways, including application of wet heat, dry heat, irradiation and heat generated by an electrical source. In several seconds to minutes, depending on the heat source used, the polymer material will soften once the temperature of the material exceeds the glass-transition temperature. While the polymer material is soft and pliable, the device is placed over the injured area, molded to conform the patient and allowed to cool. In the case of a fracture, the fracture may be placed in the corrected alignment and held while the material cools and hardens.

If the immobilization device is too large in some areas, the device may be trimmed by cutting away one or more rows of the small openings 130, 230, 330, 430 and 530 extending along the periphery of the device. These areas may be trimmed before, during or after the polymer material has cooled and set (i.e., converted back to the rigid state). The fasteners 140, 240, 340, 440 and 540 may also be placed before, during or after the polymer material has cooled and set. If desired for reinforcement, one or more peripheral edges of the immobilization device may be rolled over onto itself while the polymer material is soft to add reinforcement to the edge.

If the immobilization device needs to be adjusted (e.g., if the fracture needs to be re-aligned, or if the device rubs or creates a contact pressure point on the patient), the at least a portion of the polymer material of the immobilization device may be reheated back to a pliable state and readjusted. In some embodiments, the immobilization device may be adjusted while the device remains on the patient by applying localized heat to one or more areas of the device. For example, a hot air gun, an irradiation source (e.g., an ultrasound source or a light source) or an electrical current may be used to heat certain areas of the device past the glass transition point (T_{g}) of the polymer material, so that these areas may be reheated and re-molded onto the patient. In some embodiments, the immobilization device may be removed from the patient and placed in a hot water bath, a convection oven or a microwave for a length of time and temperature sufficient to soften all areas of the immobilization device if the entire device needs to be reformed.

It will be appreciated to those skilled in the art having the benefit of this disclosure that this invention is believed to provide a pre-fabricated, yet highly customizable immobilization device that can be configured to a patient's body habitus and/or injury specifications without requiring an expert or a lengthy amount of time to create and custom fit the device. As noted above, the immobilization device may be molded directly on the patient, and may be remolded in whole or in part, if it is determined that the immobilization devices needs adjustment. Various embodiments of immobilization devices are illustrated herein as including an arm and/or forearm splint 100, a hand and/or wrist splint 200, an ankle and/or leg splint 300, a torso splint or back brace 400, and a helmet 500. In some cases, the immobilization devices described herein may be used to treat fractures or other injuries requiring immobilization. In other cases, the immobilization devices may be used as protective gear, which an athlete may use to prevent injury or to protect an injury before it is completely healed.

It is noted that while the immobilization device described herein is illustrated for use with human patients, embodiments for veterinary use are also contemplated and included herein. For example, a pre-fabricated, yet highly customizable immobilization device may be configured to treat fractures and other injuries requiring immobilization in a variety of different animals, or in some cases, to prevent injuries from occurring. In one particular example, an immobilization device may be provided to create jumping boots for horses, which protect the horse's tendons when they land after a jump. Other uses for the immobilization device described herein may become apparent to a skilled artisan upon reading this disclosure. Since the immobilization device described herein can be pre-fabricated in any shape or size, custom fit and trimmed, the immobilization devices described herein are not limited to any particular use, body part or patient (including human and animal patients).

Further modifications and alternative embodiments of various aspects of the invention will be apparent to those skilled in the art in view of this description. It is to be understood that the immobilization devices shown and described herein are to be taken as the presently preferred embodiments. Elements and materials may be substituted for those illustrated and described herein, parts and processes may be reversed, and certain features of the immobilization devices may be utilized independently, all as would be apparent to one skilled in the art after having the benefit of this disclosure. It is intended, therefore, that the following claims be interpreted to embrace all such modifications and changes and, accordingly, the specification and drawings are to be regarded in an illustrative rather than a restrictive sense.

What is claimed is:

1. An immobilization device, comprising:
a pre-fabricated shape configured to conform to a particular body part of a patient for immobilizing that body part, wherein the pre-fabricated shape comprises a polymer material that changes from a rigid state to a pliable state upon application of heat to the polymer material;
one or more stabilization regions of solid polymer material configured to be positioned adjacent to the body part of the patient to be immobilized; and
one or more areas formed adjacent to the one or more stabilization regions, wherein the one or more areas comprise one or more large openings formed within the polymer material.

2. The immobilization device as recited in claim 1, wherein the polymer material changes from the rigid state to the pliable state when the temperature of the polymer material exceeds a transition temperature of the polymer material, and wherein the transition temperature of the polymer material ranges between approximately 100° F. and approximately 180° F.

3. The immobilization device as recited in claim 1, wherein the transition temperature of the polymer material ranges between approximately 110° F. and approximately 140° F.

4. The immobilization device as recited in claim 1, further comprising a plurality of wires or a conductive sheet, which is embedded within the polymer material and configured to receive an electrical current for applying heat to the polymer material.

5. The immobilization device as recited in claim 1, wherein the polymer material comprises a thermoplastic material or a vitrimer material.

6. The immobilization device as recited in claim 1, wherein the polymer material comprises a modified acrylonitrile butadiene styrene (ABS) material, which is synthesized with an alkyl-substituted styrene.

7. The immobilization device as recited in claim 1, wherein the pre-fabricated shape is initially provided in a flat form prior to a first application of heat to change the polymer material from the rigid state to the pliable state.

8. The immobilization device as recited in claim 1, wherein the one or more stabilization regions each comprise an area of solid polymer material ranging between about 1 inch to about 4 inches in width, and ranging between about 2 inches to about 16 inches in length, depending on the body part to be immobilized.

9. The immobilization device as recited in claim 1, wherein the one or more large openings range between about 2 mm to about 150 mm in diameter.

10. The immobilization device as recited in claim 1, wherein the immobilization device further comprises a plurality of small openings formed within the polymer material, wherein the plurality of small openings extend along a periphery of the immobilization device, and wherein the plurality of small openings enable the immobilization device to be trimmed and also provide a plurality of attachment locations for securing the immobilization device onto the patient with fasteners.

11. The immobilization device as recited in claim 10, wherein each of the plurality of small openings range between about 3 mm to about 30 mm in length and about 2 mm to about 15 mm in width, and wherein the small openings are spaced from each other by about 2 mm to about 15 mm.

12. The immobilization device as recited in claim 10, wherein the plurality of small openings extend along an entire periphery of the immobilization device.

13. The immobilization device as recited in claim 10, wherein the immobilization device comprises multiple rows of the plurality of small openings along one or more peripheral edges of the immobilization device.

14. The immobilization device as recited in claim 1, wherein the pre-fabricated shape is formed via an extrusion, injection molding, compression molding or 3-D printing process.

15. The immobilization device as recited in claim 1, wherein the pre-fabricated shape is stamped or cut from a flat sheet of the polymer material.

16. A method for fitting an immobilization device onto a patient, comprising:
obtaining an immobilization device having a pre-fabricated shape, which is rigid, yet configurable to conform to a body part of the patient through application of heat to the immobilization device, wherein the immobilization device comprises a polymer material having a transition temperature ranging between approximately 100° F. and approximately 180° F.;
applying heat to the immobilization device to increase a temperature of the polymer material past the transition temperature and convert the polymer material to a pliable state; and
conforming the immobilization device to the body part while the polymer material is in the pliable state, and subsequently allowing the polymer material to cool and harden back to a rigid state to immobilize the body part.

17. The method as recited in claim 16, wherein the step of applying heat to the immobilization device comprises placing the immobilization device in a hot water bath, a convection oven or a microwave oven for a length of time and temperature needed to increase the temperature of the polymer material past the transition temperature.

18. The method as recited in claim 16, wherein the step of applying heat to the immobilization device comprises using a hot air gun, an ultrasound source, an electrical source or a light source to apply heat to the immobilization device for a length of time and temperature needed to increase the temperature of the polymer material past the transition temperature.

19. The method as recited in claim 16, further comprising determining if adjustment is needed in at least a portion of the immobilization device, wherein if adjustment is needed, the method further comprises:
reapplying heat to at least the portion of the immobilization device, while the immobilization device remains on the patient, to convert the polymer material back to the pliable state; and
reforming the at least portion of the immobilization device and subsequently allowing the polymer material to cool and harden back to the rigid state.

20. The method as recited in claim 19, wherein the step of reapplying heat comprises using a hot air gun, an ultrasound source, an electrical source or a light source to reapply heat locally to at least the portion of the immobilization device determined to need adjustment.

21. The method as recited in claim 16, wherein the immobilization device comprises one or more rows of small openings extending along an entire periphery of the immobilization device.

22. The method as recited in claim 21, further comprising tailoring the immobilization device to fit a particular body part or patient by cutting away one or more of the rows of small openings.

23. The method as recited in claim 21, further comprising using a plurality of the small openings for attaching fasteners at multiple places along the periphery of the immobilization device to fasten the immobilization device onto the patient.
